(19) United States
(12) Patent Application Publication
(10) Pub. No.: US 2022/0193140 A1
(43) Pub. Date: Jun. 23, 2022

(54) WASHED PLATELET EXTRACT

(71) Applicant: WISCONSIN ALUMNI RESEARCH FOUNDATION, Madison, WI (US)

(72) Inventors: Jacques GALIPEAU, Madison, WI (US); Sabrina Helen BROUNTS, Verona, WI (US); Andrea PENNATI, Madison, WI (US)

(21) Appl. No.: 17/609,064

(22) PCT Filed: May 8, 2020
(86) PCT No.: PCT/US2020/032018

§ 371 Date: Nov. 5, 2021

Related U.S. Application Data
(60) Provisional application No. 62/845,140, filed on May 8, 2019.
(60) Provisional application No. 62/971,425, filed on Feb. 7, 2020.

(51) Int. Cl. A61K 35/19; A61K 9/19; A61P 37/06

(52) U.S. Cl. CPC A61K 35/19; A61P 37/06; A61K 9/19

(57) ABSTRACT

A washed platelet extract with increased platelet growth factor concentration and decreased total protein relative to platelet rich plasma, and methods of making and using such an extract for the treatment of disease and injuries recited herein, are provided.

### CROSS-REFERENCE TO RELATED APPLICATIONS

This application claims priority to U.S. Provisional Application No. 62/845,140, filed May 8, 2019, and U.S. Provisional Application No. 62/971,425, filed Feb. 7, 2020, each of which is incorporated herein by reference in its entirety.

### STATEMENT REGARDING FEDERALLY SPONSORED RESEARCH

Not applicable.

### REFERENCE TO A SEQUENCE LISTING SUBMITTED VIA EFS-WEB

Not applicable.

### BACKGROUND

Experimental and clinical data established platelets to be critical in physio-pathological processes such as inflammation, tissue regeneration and revascularization [1]. Regenerative growth factors and cytokines contained in platelets have always been appealing in regenerative and sports medicine [2]. In vivo, platelets are activated at the site of injury and release cytokines and growth factors stored in the dense membrane of the a-granules [3]. These molecules, when bound to the proper receptors, promote expression of genes involved in cellular recruitment, growth and morphogenesis [4]. Other molecules present in the a-granules (e.g., adhesive proteins, clotting factors, fibrinolytic factors etc.) are involved in the inflammation processes [4]. In general, activation of platelets allows the release of a large number of soluble mediators, that are essential in wound healing, clotting, and angiogenesis [1-3]. Hence the interest in using platelets or blood plasma derivatives for regenerative medicine and tissue engineering applications.

In the past 40 years, the possibility of using platelet or blood plasma derivatives has been extensively investigated [4, 5]. The first whole blood-derived product was platelet rich plasma (PRP) and used to describe the plasma with a platelet count above that of peripheral blood [6]. Ten years later, autologous PRP was used in maxillofacial surgery for its high fibrinogen content and anti-inflammatory characteristics able to induce cell proliferation [7]. Later on, autologous PRP has been used predominantly in the musculoskeletal field in sport injuries [6]. Other fields of PRP use include cardiac surgery, pediatric surgery, gynecology and more recently dermatology [6, 8-11].

A later formulation aimed to improve the potency of PRP was the so-called platelet lysate (PL) obtained by a simple step of freezing and thawing PRP with the intent of disrupting platelets contained therein and further centrifugation to remove any residual platelet fragments [5, 6]. More recently, different methods have been validated to remove fibrinogen from the lysate without compromising the final growth factors and cytokine composition [12, 13].

Allogeneic and autologous PRP have been tested in human medicine for tendinopathies like rotator cuff repair [14, 15], wound healing in ophthalmology [16], non-healing ulcers [17], persistent fracture non-unions [18], adhesive capsulitis of the shoulder (NCT01458691), endometrial regeneration (NCT02825849), as well PL for epicondylitis [19] and cornea and ocular surface diseases [20]. Similarly, PRP has been used in veterinary medicine for the treatment of sport related injuries in horses for the rehabilitation of tendinous and ligamentous injuries [21-23], and wound healing [24]. Unfortunately, other plasma proteins are still present in the final formulation, and not all the components stored in the platelets can be efficiently released by the freezing-thawing procedure. Hence, only limited clinical results have been obtained, either in human medicine or veterinary medicine, with PRP and PL [21, 25-27].

Roadblocks to the use of PRP and PL include the large the amount of donor serum required, the low concentration of growth factors and cytokines, and the possibility of allograft material rejection [28]. In some cases, the volume of serum that needs to be injected into an injection site is still too large for the anatomical region, causing a less than ideal amount of serum injected to treat the injury being used. Furthermore, reactions can be caused using this method due to incompatibility between donor and recipient, as well as transfer of diseases [28, 29].

Therefore, a need in the art exists for improved platelet compositions, including compositions for autologous, xenogeneic, allogeneic, or syngeneic applications, for use in various treatment methods without the risk for serum sickness or allergic reaction side effects.

### SUMMARY OF THE INVENTION

In a first aspect, provided herein is a method of producing a washed platelet extract comprising the steps of i) separating plasma from a whole blood sample; ii) separating platelets from the plasma; iii) washing the platelets with buffered saline; iv) adding a detergent solution comprising between 0.1% and 2.5% detergent to the washed platelets and incubating the washed platelets with the detergent solution for a time sufficient to lyse at least 50% of the platelets, whereby a platelet lysate is formed; and v) separating the washed platelet extract from insoluble components of the platelet lysate. In some embodiments, the method additionally comprises the step of removing residual detergent from the washed platelet extract by spinning or using hydrophobic interaction chromatography. In some embodiments, the method additionally comprises the step of lyophilizing the washed platelet extract to form a lyophilized washed platelet extract. In some embodiments, in step (i), the plasma is separated from the whole blood sample by spinning the whole blood sample at a speed between 600×g and 1000×g for about 5 minutes at a temperature between 22° C. and 27° C. In some embodiments, in step (ii), the platelets are separated from the plasma by spinning the plasma at a speed between 600×g and 1000×g for about 25 minutes at a temperature between 22° C. and 27° C. In some embodiments, the buffered saline is phosphate buffered saline (PBS). In some embodiments, in step (iii), the platelets are washed at least 2 times. In some embodiments, in step (v), the washed platelet extract is separated from the insoluble components by spinning the platelet lysate at a speed between 3000×g and 5000×g for about 10 minutes at about 4° C. In some embodiments, prior to step (ii) the plasma is stored for at least 1 day at 4° C. In some embodiments, the washed platelets are incubated with the detergent solution for at least 1 hour. In some embodiments, the whole blood sample is an equine whole blood sample and the washed platelet extract produced is a washed equine platelet extract.

In some embodiments, the detergent selected from the group consisting of (1,1,3,3-Tetramethylbutyl)phenyl-polyethylene glycol (TRITON™ X-114), nonyl phenoxypolyethoxylethanol (NP-40), 3-[(3 -Cholamidopropyl)dimethylammonio]-1-propanesulfonate (CHAPS), or polyoxyethylene (20) sorbitan monolaurate (TWEEN 20). In some embodiments, the detergent solution comprises 2% (1,1,3,3-Tetramethylbutyl)phenyl-polyethylene glycol.

In a second aspect, provided herein is a washed platelet extract generated by the methods described herein and free of plasma serum protein. In some embodiments, the washed platelet extract comprises at least 20 ng/mL of platelet growth factors when prepared from 50 mL of plasma. In some embodiments, the platelet growth factors include platelet derived growth factor (PDGF). In some embodiments, the washed platelet extract comprises less than 2 mg/mL total protein when prepared from 50 mL of plasma. In some embodiments, the washed platelet extract is lyophilized.

In a third aspect, provided herein is a pharmaceutical composition comprising the washed platelet extract described herein and a pharmaceutically acceptable carrier.

In a fourth aspect, provided herein is a method of treating lameness in a horse in need thereof, comprising administering to the horse a therapeutically effective amount of a pharmaceutical composition of washed equine platelet extract described herein, wherein the whole blood sample is an equine whole blood sample. In some embodiments, the pharmaceutical composition is administered at a volume between 0.1 mL and 1.0 L.

In a fifth aspect, provided herein is a method of treating tendon or ligament injury in a subject in need thereof, comprising administering to the subject a therapeutically effective amount of a pharmaceutical composition of washed platelet extract described herein. In some embodiments, the subject is a human, a horse, a dog, a cat, a camelid, or a cow.

### INCORPORATION BY REFERENCE

All publications, patents, and patent applications mentioned in this specification are herein incorporated by reference to the same extent as if each individual publication, patent, and patent application was specifically and individually indicated to be incorporated by reference.

### BRIEF DESCRIPTION OF DRAWINGS

FIGS. 1A-1B show platelet isolation and lysis from equine whole blood samples. FIG. 1A shows separation of platelets from plasma following centrifugation of plasma at 800×g for 25 minutes at room temperature. FIG. 1B shows separation of the washed equine platelet extract from insoluble components in the platelet lysate following lysis with 2% Triton-X114 and centrifugation at 800×g for 10 minutes at 4° C.

FIG. 2 shows GFP/IL-10 expression in M2-like macrophage cells. Live cells were selected based on Ghost-red^{dim} fluorescence. Macrophages were selected for double positive expression of CD45⁺CD11b⁺. Flow cytometry parameters for the GFP⁺/IL10⁺cells were previously established using C57BL/6 mice.

FIG. 3 shows the effects of intraperitoneal (IP) injection of the washed equine platelet extract (fresh or lyophilized and reconstituted) or PBS in the dextran sulfate sodium (DSS)-induced colitis model. C57BL/6 mice were challenged with 4% DSS in the drinking water for 7 days to induce the disease followed by a recovery phase with water. Mice received IP injection of the washed equine platelet extract (1 mL of fresh or lyophilized and reconstituted) or PBS (1 mL) at day 2 and 4. Body weight was determined daily for each mouse and is depicted as the percentage of the initial value measured before the onset of the experiment (n=5 in each group). Data are expressed as the mean ±SEM and analyzed by Student's t-test.

FIG. 4 shows an equine T cell suppression assay testing the effects of WEPLEX on T cell proliferation. Equine peripheral blood mononuclear cells (PBMCs) were isolated from whole blood, stained with CFSE and stimulated with PHA. After 3 days, the cells were collected and stained for CD8. With increasing concentration of WEPLEX the T cells proliferation decreased.

FIG. 5 shows a human T cell suppression assay testing the effects of WEPLEX on T cell proliferation. Human PBMCs were isolated from whole blood and stimulated with PHA. After 3 days the cells were collected and stained for Ki-67 and CD3. With increasing concentration of WEPLEX the T cells proliferation decreased (12.5% compare to 0.7%, upper right square).

FIG. 6 shows a comparison of T cells (equine and human PBMCs) following treatment with fresh and lyophilized/reconstitute WEPLEX in a T cell suppression assay. Statistical analysis has been carried out with 3 replicates and 3 independent preparations of WEPLEX and the lyophilized/reconstituted WEPLEX. Data are expressed as the mean±SD and analyzed by Student's t-test, *P<0.05, **P<0.01, ***P<0.001

### DETAILED DESCRIPTION OF THE DISCLOSURE

The present disclosure broadly relates to a washed platelet extract as well as methods of making and using such an extract. In some embodiments, the washed platelet extract is a washed equine platelet extract.

In one aspect of the invention, whole blood samples are centrifuged, the top plasma layer is removed and centrifuged again to form a platelet pellet, the platelet pellet is washed, lysed with detergent, and centrifuged to yield a washed platelet extract. The washed platelet extract, preferably generated by the methods described herein, may be used to treat a disease or injury by administration of the washed platelet extract to a subject in need thereof. References herein to centrifugation or spinning are considered equivalent terms.

As used herein, “washed platelet extract” refers to a product derived from whole blood and having a high concentration of growth factors and cytokines but free of immunogenic serum plasma proteins. Growth factors and cytokines of the washed platelet extract include, but are not limited to, platelet derived growth factor (PDGF), transforming growth factor-beta1 (TGF-β1), insulin-like growth factor-I (IGF-I), platelet-derived growth factor-AB (PDGF-AB), PDGF-BB, vascular endothelial growth factor (VEGF), epidermal growth factor (EGF), IL-4, IL-5, IL-6, IL-8, IL-12, IL-13, IL-17, INF-γ, TNF-α, MCP-1, MIP-1a, RANTES, IL-2, IL-5, IL-7, IL-9, IL-10, IL-15 G-CSF, GM-CSF, eotaxin, CXCL10 chemokine (IP-10), and MIP lb. A high concentration of growth factors and cytokines is considered to be a total growth factor and cytokine concentration of at least 10 ng/mL, at least 12 ng/mL, at least 15 ng/mL, at least 20 ng/mL, at least 25 ng/mL, or at least 30 ng/mL in a washed platelet extract produced from 50 mL of plasma. In some embodiments, the washed platelet extract includes at least 20 ng/mL of PDGF when produced from 50 mL of plasma. In some embodiments, the washed platelet extract includes at least 30 ng/mL of PDGF when produced from 50 mL of plasma. In some embodiments, the total protein concentration in the washed platelet extract is less than 2 mg/mL, less than 1.5 mg/mL, less than 1 mg/mL, less than 0.8 mg/mL, less than 0.6 mg/mL, or less than 0.5 mg/mL when the washed platelet extract is produced from 50 mL of plasma. The washed platelet extract may be produced from any starting volume of whole blood or plasma as described herein. For example, when the washed platelet extract is produced from 100 mL, 150 mL, 200 mL, 250 mL, or 500 mL of plasma, the concentrations of growth factors, cytokines, and total protein may be 2×, 3×, 4×, 5×, or 10× higher than those specifically recited herein when the final volume of washed platelet extract is the same.

The whole blood or plasma used to produce the washed platelet extract may be from any suitable source, including, but not limited to, equine, bovine, human, non-human primate, porcine, ovine, caprine, feline, canine, camelid, and other mammals. In some embodiments, the whole blood is equine whole blood and the washed platelet extract produced is a washed equine platelet extract. Equine whole blood and platelets are easily acquired in relatively large quantities compared to, for example, human whole blood or platelets. Additionally, equine whole blood and platelets are preferentially used rather than whole blood and platelets of other mammalian species, as the equine components are free of transmissible zoonotic diseases. However, the washed platelet extracts described herein may be prepared from any species and used for autologous, syngeneic, allogeneic, xenogeneic, or third party treatment, as would be understood by one of skill in the art in possession of the disclosure.

As used herein, “washed equine platelet extract” refers to a product derived from equine blood with a high concentration of growth factors and cytokines, such as platelet derived growth factor (PDGF), but free of immunogenic serum plasma proteins.

As used herein, “free” refers to a composition or conditions devoid of a certain component or reagent. Free of immunogenic serum proteins means the composition contains less than 1% of total immunogenic serum proteins, or less than 0.5% of total immunogenic serum proteins by weight, and preferably contains less than 0.4%, less than 0.2%, less than 0.1% or less than 0.05% of total immunogenic serum proteins. Immunogenic serum proteins are proteins not recognized by the host organism that are antigenic and elicit an immune response in the host. Immunogenic serum proteins may include, but are not limited to albumin, immunoglobulins, fibrinogen, and complement. In some embodiments, the washed platelet extract includes less than 0.2% of albumin, immunoglobulins, fibrinogen, or complement.

### Platelet Processing Methods

A whole blood sample may be collected from any suitable source or any suitable species. The whole blood sample may be mixed with a citrate anticoagulant. In some embodiments, the whole blood sample is stored in a citrate phosphate dextrose adenine (CPDA-1) bag. In some embodiments, the whole blood is human whole blood. In some embodiments, the whole blood is equine whole blood.

An equine whole blood sample is collected from a horse. The horse may be any individual of the species Equus ferus, including the subspecies Equus ferus caballus. The equine whole blood sample may be mixed with a citrate anticoagulant. The maximum donation volume per equine animal is typically 15-18 mL/kg body weight every 3-4 weeks. The volume of equine whole blood collected from the individual may be at least .5 L, at least 1 L, at least 1.5 L, at least 2 L, at least 3 L, at least 4 L, at least 5 L, at least 6 L, at least 7 L, or at least 7.5 L. In some embodiments, the equine whole blood sample is stored in a CPDA-1 bag.

The whole blood is first processed by spinning for a time and under conditions sufficient to separate the whole blood into three layers after spinning—(i) the plasma (top layer); (ii) the white blood cells (middle layer); and (iii) the red blood cells (bottom layer). The whole blood may be centrifuged at a speed between 600×g and 1000×g (e.g., 600×g, 650×g, 700×g, 750×g, 800×g, 850×g, 900×g, 950×g, or 1000×g). In some embodiments, the whole blood may be centrifuged without the brake, such that the spinning is stopped without mechanical assistance. The whole blood may be centrifuged at a temperature between 20° C. and 27° C. (e.g., 20° C., 21 ° C., 22° C., 23° C., 24° C., 25° C., 26° C., or 27° C.). The blood may be spun or centrifuged for about 10 minutes (e.g., 3 minutes, 4 minutes, 5 minutes, 6 minutes, 7 minutes, 8 minutes, 9 minutes, or 10 minutes.). In some embodiments, the whole blood is spun at 800×g for 5 minutes at 25° C. without braking to reduce speed. In some embodiments, whole blood is stored at 4° C. for up to 24 hours prior to processing.

Following separation of the whole blood, the top plasma layer is removed and transferred to a separate container. The plasma may be stored at a temperature of about 4° C. for about 1 day, about 2 days, about 3 days, about 4 days, about 5 days, about 6 days, about 7 days or about 8 days. Without wishing to be bound by any particular theory, storage of the separated plasma for such a period of time prior to separation of the platelets from the plasma reduces platelet aggregation and improves resuspension and washing of the platelets. In general, lysis yield from stored platelets is improved over lysis yield from platelets immediately processed.

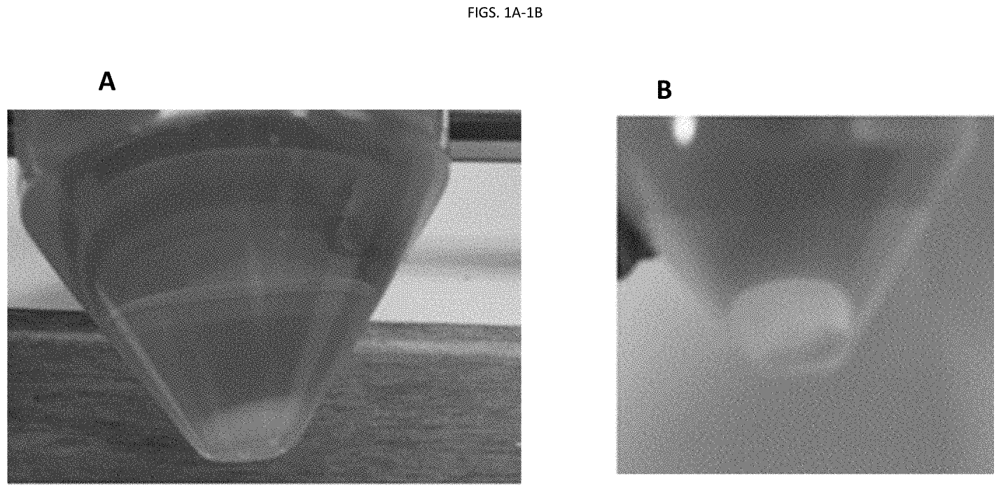

After storage, plasma is processed by spinning or centrifugation for a time and under conditions sufficient to separate the platelets from the serum plasma (FIG. 1A). The plasma may be centrifuged at a speed between 600×g and 1000×g (e.g., 600×g, 650×g, 700×g, 750×g, 800×g, 850×g, 900×g, 950×g, or 1000×g). The plasma may be centrifuged at a temperature between 20 ° C. and 27° C. (e.g., 20° C., 21° C., 22° C., 23° C., 24° C., 25° C., 26° C., or 27° C.). The plasma may be centrifuged for about 25 minutes (e.g., 20 minutes, 21 minutes, 22 minutes, 23 minutes, 24 minutes, 25 minutes, 26 minutes, 27 minutes, 28 minutes, 29 minutes, or 30 minutes). In some embodiments, the plasma is spun at 800×g for 25 minutes at 25° C.

Following separation of the platelets from the serum, the serum is removed. The resulting platelet pellet is washed with buffered saline (e.g., phosphate buffered saline (PBS)) to remove residual serum and serum proteins. The washing step includes resuspension of the platelet pellet with buffered saline, followed by pelleting the platelets and removing the wash buffer. In some embodiments, the platelets are washed at least twice with PBS. In some embodiments, the platelets are washed at least three times with PBS. After the platelets are washed and pelleted, they may be stored at −80° C. In some embodiments, platelets may be stored for a day, a week, a month, 3 months, 6 months, 9 months, one year or more before lysis. In some embodiments, the platelets are lysed immediately after washing.

To lyse the platelets, platelets are resuspended in a detergent solution for a time sufficient for at least 50% (e.g., at least 60%, at least 70%, at least 75%, at least 80%, at least 85%, at least 90%, at least 95%, at least 97%, at least 98%, at least 99%, or at least 99.9%) of platelets to lyse. The platelets may be exposed to the detergent solution for at least 1 hour, at least 2 hours, at least 4 hours, at least 6 hours, at least 8 hours, at least 12 hours, at least 18 hours or at least 20 hours. The detergent solution may include between about 0.1% and about 2.5% detergent, or between about 1% and about 2% detergent. The detergent solution may include 0.1%, 0.5%, 1%, 1.5%, 2% or 2.5% detergent. In some embodiments, the detergent solution includes a buffered saline, such as PBS. In some embodiments, the detergent solution includes 2% Triton-X114. In some embodiments, the detergent solution includes 1% Triton-X114. In some embodiments, the detergent solution includes 1% CHAPS. In some embodiments, the detergent solution includes 1% TWEEN 20. In some embodiments, the detergent solution includes 1% NP-40.

A suitable detergent for use in the platelet lysis step may be any non-denaturing detergent that can subsequently be removed from the platelet lysate. In some embodiments, the detergent is be selected from the group consisting of (1,1,3,3-Tetramethylbutyl)phenyl-polyethylene glycol (TRITONTM X-114), nonyl phenoxypolyethoxylethanol (NP-40), 3-[(3-Cholamidopropyl)dimethylammonio]-1-propanesulfonate (CHAPS), or polyoxyethylene (20) sorbitan monolaurate (TWEEN 20). Some non-denaturing detergents may be removed from the resulting platelet lysate more easily and efficiently than others. Selection of a readily removable detergent is preferred, but any non-denaturing detergent that can be separated from the lysate by any means is suitable. Detergent removal is outlined below.

In some embodiments, the detergent is Triton-X114. Triton-X114 provides the added advantage of being removable by centrifugation due to its cloud point at 22° C. Additionally, the use of Triton-X114 allows removal of endotoxin from the washed platelet extract. In some embodiments, the washed platelet extract is prepared using Triton-X114 and is substantially free of endotoxin.

After the platelets are lysed to yield a platelet lysate, the platelet lysate is centrifuged at a time and under conditions sufficient to separate from the platelet lysate a soluble fraction, referred to herein as the washed platelet extract, from the insoluble lysate components of the platelet lysate. Insoluble lysate components may include platelet cell membranes and other insoluble components such as organelle membranes. The platelet lysate may be centrifuged at a speed between 3000×g and 6000×g (e.g., 3000×g, 3250×g, 3500×g, 3750×g, 4000×g, 4250×g, 4500×g, 4750×g, 5000×g, 5250×g, 5500×g, 5750×g, or 6000×g). The platelet lysate may be centrifuged at about 4° C. (e.g., 2° C., 3° C., 4° C., 5° C., or 6° C.). The platelet lysate may be spun for about 10 minutes (e.g., 5 minutes, 6 minutes, 7 minutes, 8 minutes, 9 minutes, 10 minutes, 11 minutes, 12 minutes, 13 minutes, 14 minutes, or 15 minutes). In some embodiments, the platelet lysate is spun at 4000×g for about 10 minutes at 4° C. to form the washed platelet extract which is separated from the insoluble lysate components.

In some embodiments, following separation of the washed platelet extract from the insoluble lysate components, detergent is removed from the washed platelet extract. Detergent may be removed by any suitable means known in the art including, but not limited to, centrifugation, direct binding, gel filtration, dialysis, precipitation, and ion-exchange chromatography. The most appropriate method will depend on the molecular weight, concentration, and other properties of the detergent used for platelet lysis. For example, some detergents may have cloud points suitable for removal using centrifugation. Some detergents may be removed using detergent removal spin columns such as DetergentOUT™. In general, detergent properties and suitable methods for the removal thereof are known in the art, even while selection of a particular detergent to achieve the goals of the inventive methods is not.

In some embodiments, the washed platelet extract is additionally spun or centrifuged to remove residual detergent. The washed platelet extract may be centrifuged at a speed between 12,000×g and 17,000×g (e.g., 12,000×g, 13,000×g, 14,000×g, 15,000×g, 16,000×g, or 17,000×g). The washed platelet extract may be centrifuged at a temperature between 23° C. and 30° C. (e.g., 23° C., 24° C., 25° C., 26° C., 27° C., 28° C., 29° C., or 30° C.). The washed platelet extract may be centrifuged for about 20 minutes (e.g., 10 minutes, 11 minutes, 12 minutes, 13 minutes, 14 minutes, 15 minutes, 16 minutes, 17 minutes, 18 minutes, 19 minutes, or 20 minutes). In some embodiments, the washed platelet extract is further processed by hydrophobic interaction chromatography to remove residual detergent.

In some embodiments, the washed platelet extract is lyophilized to form a solid powdered platelet extract composition. This lyophilized washed platelet extract is suitable for storage, for example, at −80° C. for at least 1 day, at least 1 week, at least 2 weeks, at least 1 month, at least 2 months, at least 6 months, or at least 1 year. The lyophilized washed platelet extract may be reconstituted in in any volume of water or buffer prior to use. Reconstitution of the lyophilized washed platelet extract in reduced volumes of water or buffer allows for production of a more concentrated washed platelet extract product.

### Treatment

According to the methods of the present disclosure, washed platelet extract is administered to a subject in need thereof. Subjects in need of treatment include those already having or diagnosed with a disease or injury as described herein, or those who are at risk of developing a disease or injury as described herein.

A disease or injury of the present disclosure may include, but is not limited to, lameness, tendon injuries, degenerative joints, ligament injuries, bursitis, osteoarthritis, degenerative joint disease, inflammatory joint conditions, septic joints, tendon sheath injuries, tendon sheath inflammation, soft tissue injuries (including musculoskeletal soft tissue injuries), meniscal injuries, muscle injuries, bone fractures, fracture repair non-union, delayed unions or mal-unions in fracture healing, bone grafting, bone cysts, wounds, wound healing, wound infection, skin grafting, skin ulcers, burns, dry eye, corneal ulcers, conjunctival flap grafting, inflammation of the eye globe/orbit or the surrounding structures and tissues, gastrointestinal tract injury, gastrointestinal tract inflammation, septicemia, bacteremia, tooth decay, tooth extraction, surgical infection, spinal and nerve inflammation, and cancer (e.g., bladder cancer).

Advantageously, the methods provided herein improve the chance for the subject to receive therapeutic benefit from a platelet therapy while minimizing the risk for life threatening complications from serum sickness or allergic reactions associated with the presence of immunogenic serum proteins (e.g., albumin, fibrinogen, complement, and IgG).

As used herein, the terms “treat” and “treating” refers to therapeutic measures, wherein the object is to slow down (lessen) an undesired physiological change or pathological disorder resulting from a disease or injury as described herein. For purposes of this invention, treating the disease or injury includes, without limitation, alleviating one or more clinical indications, decreasing inflammation, reducing the severity of one or more clinical indications of the disease or injury, diminishing the extent of the condition, stabilizing the subject's disease or injury (i.e., not worsening), delay or slowing, halting, or reversing the disease or injury and bringing about partial or complete remission of the disease or injury. Treating the disease or injury also includes prolonging survival by days, weeks, months, or years as compared to prognosis if treated according to standard medical practice not incorporating treatment with the washed platelet extract.

Subjects in need of treatment can include those already having or diagnosed with a disease or injury as described herein as well as those prone to, likely to develop, or suspected of having a disease or injury as described herein. Pre-treating or preventing a disease or injury according to a method of the present invention includes initiating the administration of a therapeutic (e.g., washed platelet extract) at a time prior to the appearance or existence of the disease or injury, or prior to the exposure of a subject to factors known to induce the disease or injury. Pre-treating the disorder is particularly applicable to subjects at risk of having or acquiring the disease injury. As used herein, the terms “prevent” and “preventing” refer to prophylactic or preventive measures intended to inhibit undesirable physiological changes or the development of a disorder or condition resulting in the disease or injury. In exemplary embodiments, preventing the disease or injury comprises initiating the administration of a therapeutic (e.g., washed platelet extract) at a time prior to the appearance or existence of the disease or injury such that the disease or injury, or its symptoms, pathological features, consequences, or adverse effects do not occur. In such cases, a method of the invention for preventing the disease or injury comprises administering washed platelet extract to a subject in need thereof prior to exposure of the subject to factors that influence the development of the disease or injury. In some embodiments, the subject is a racehorse and the administration of washed platelet extract is intended to prevent degeneration of joints. In some embodiments, washed platelet extract is coated on a surgical implant to prevent infection. In some embodiments, washed platelet extract is administered to a subject for skin rejuvenation.

As used herein, the terms “subject” or “patient” are used interchangeably and can encompass any vertebrate including, without limitation, humans, mammals, reptiles, amphibians, and fish. However, advantageously, the subject or patient is a mammal such as a human or non-human primate, or a mammal such as a domesticated mammal, e.g., dog, cat, horse, and the like, or livestock, e.g., cow, sheep, pig, and the like. In some embodiments, the subject is a horse. In some embodiments, the subject is a human. As used herein, the phrase “in need thereof” indicates the state of the subject, wherein therapeutic or preventative measures are desirable. Such a state can include, but is not limited to, subjects having a disease or injury as described herein or a pathological symptom or feature associated with a disease or injury as described herein. Washed platelet extract can be autologous, xenogeneic, syngeneic, allogeneic, or third party with respect to the subject or patient being treated.

In some cases, a method of treating or preventing a disease or injury as described herein comprises administering a pharmaceutical composition comprising a therapeutically effective amount of washed platelet extract as a therapeutic agent (i.e., for therapeutic applications). As used herein, the term “pharmaceutical composition” refers to a chemical or biological composition suitable for administration to a mammal. Examples of compositions appropriate for such therapeutic applications include preparations for oral, topical, parenteral, subcutaneous, transdermal, intradermal, intramuscular, intraperitoneal, intraocular, intravenous (e.g., injectable), intraparenchymal, intraarticular, intraosseous, intratendinous, intraligamentous, subconjunctival, intrathecal, epidural, intracardial, or intraarterial administration. In some embodiments, the composition is applied topically to the eye as a cream or spray. In some cases, pharmaceutical compositions appropriate for therapeutic applications may be in admixture with one or more pharmaceutically acceptable excipients, diluents, or carriers such as sterile water, physiological saline, glucose or the like. For example, washed platelet extract described herein can be administered to a subject as a pharmaceutical composition comprising a carrier solution.

Formulations may be designed or intended for oral, rectal, nasal, topical or transmucosal (including buccal, sublingual, ocular, vaginal and rectal) and parenteral (including subcutaneous, intramuscular, intravenous, intradermal, intraperitoneal, intrathecal, intraarticular, intratendinous, intraocular, intraparenchymal, intrathecal and epidural) administration. In some embodiments, the formulation may be a sterile suspension, emulsion, or aerosol. In general, aqueous and non-aqueous liquid or cream formulations are delivered by a parenteral, oral or topical route. In other embodiments, the compositions may be present as an aqueous or a non-aqueous liquid formulation or a solid formulation suitable for administration by any route, e.g., oral, topical, buccal, sublingual, parenteral, aerosol, a depot such as a subcutaneous depot or an intraperitoneal, intraparenchymal or intramuscular depot. In some cases, pharmaceutical compositions are lyophilized. In other cases, pharmaceutical compositions as provided herein contain auxiliary substances such as wetting or emulsifying agents, pH buffering agents, gelling or viscosity enhancing additives, preservatives, flavoring agents, colors, and the like, depending upon the route of administration and the preparation desired. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

The preferred route may vary with, for example, the subject's pathological condition or weight or the subject's response to therapy or that is appropriate to the circumstances. The formulations can also be administered by two or more routes, where the delivery methods are essentially simultaneous or they may be essentially sequential with little or no temporal overlap in the times at which the composition is administered to the subject.

Suitable regimes for initial administration and further doses or for sequential administrations also are variable, may include an initial administration followed by subsequent administrations, but nonetheless, may be ascertained by the skilled artisan from this disclosure, the documents cited herein, and the knowledge in the art.

In some cases, washed platelet extract may be optionally administered in combination with one or more additional active agents. Such active agents include anti-inflammatory, anti-cytokine, analgesic, antipyretic, antibiotic, and antiviral agents, as well as growth factors and agonists, antagonists, and modulators of immunoregulatory agents (e.g., TNF-α, IL-2, IL-4, IL-6, IL-10, IL-12, IL-13, IL-18, IFN-α, IFN-γ, BAFF, CXCL13, IP-10, VEGF, EPO, EGF, HRG, Hepatocyte Growth Factor (HGF), Hepcidin, including antibodies reactive against any of the foregoing, and antibodies reactive against any of their receptors). In some embodiments, the washed platelet extract may be administered in combination with a steroid, hyaluronic acid, polysulfated glycosaminoglycans, antibiotics, stem cells, interleukin 1 receptor agonist, bone marrow concentrate, platelet rich plasma, or combinations thereof. In some embodiments, for bone or wound repair washed platelet extract may be administered with or incorporated within bone cement, plaster of Paris, poly(methyl methacrylate), scaffolds, sutures, or implants. Any suitable combination of such active agents is also contemplated. When administered in combination with one or more active agents, washed platelet extract can be administered either simultaneously or sequentially with other active agents. For example, a subject with a tendon or ligament injury may simultaneously receive washed platelet extract and a steroid or corticosteroid for a length of time or according to a dosage regimen sufficient to support recovery and to treat, alleviate, or lessen the severity of the tendon or ligament injury. In some embodiments, washed platelet extract may be administered to augment bone grafting treatment. In some embodiments, washed platelet extract may be administered as a coating on an implant.

In some embodiments, washed platelet extract is administered to a subject simultaneously undergoing surgical, arthroscopic, or endoscopic repair of an orthopedic injury. In such cases, washed platelet extract can be provided to a subject in need thereof in a pharmaceutical composition adapted for direct administration to the tendon or ligament. Administration may be provided before, after, or simultaneous with repair and suture of the tendon or ligament. Washed platelet extract can be administered directly to the tendon or ligament being repaired. Washed platelet extract may also be administered as part of a treatment in which the subject is receiving donor or graft tissues. Washed platelet extract may be applied via a collagen sponge or gel, hydrogel, or tissue engineered scaffold. Administration as part of a cell sheet or sheath around the tendon or ligament being treated is also envisioned. In some embodiments, sutures may be coated with the washed platelet extract.

In some embodiments, washed platelet extract is administered to a subject in need thereof using an infusion, topical application, surgical transplantation, or implantation. In an exemplary embodiment, administration is systemic. In such cases, washed platelet extract can be provided to a subject in need thereof in a pharmaceutical composition adapted for intravenous administration to subjects. Typically, compositions for intravenous administration are solutions in sterile isotonic aqueous buffer. The use of such buffers and diluents is well known in the art. Where necessary, the composition may also include a local anesthetic to ameliorate any pain at the site of the injection. Generally, the ingredients are either supplied separately or mixed together in unit dosage form, for example, as a cryopreserved concentrate in a hermetically sealed container such as an ampoule indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration. In some cases, compositions comprising washed platelet extract are cryopreserved prior to administration. In some cases, compositions comprising washed platelet extract are lyophilized and resuspended prior to administration.

Therapeutically effective amounts of washed platelet extract are administered to a subject in need thereof. An effective dose or amount is an amount sufficient to affect a beneficial or desired clinical result. With regard to methods of the present invention, the effective dose or amount, which can be administered in one or more administrations, is the amount of washed platelet extract sufficient to elicit a therapeutic effect in a subject to whom the extract is administered. In some cases, an effective dose of washed platelet extract is about 0.1 mL to about 1 L (e.g., 0.1 mL, 0.2 mL, 0.5 mL, 1 mL, 5 mL, 10 mL, 12 mL, 15 mL, 20 mL, 25 mL, 30 mL, 40 mL, 50 mL, 75 mL, 100 mL, 150 mL, 200 mL, 500 mL, 750 mL, or 1L) that includes about 10 ng/mL to about 1 mg/mL (e.g., 10 ng/mL, 20 ng/mL, 30 ng/mL, 40 ng/mL, 50 ng/mL, 75 ng/mL, 100 ng/mL, 500 ng/mL, 1000 ng/mL, 5000 ng/mL, 0.01 mg/mL, 0.05 mg/mL, 0.1 mg/mL, 0.5 mg/mL, or 1 mg/mL) total cytokines and growth factors. Effective amounts will be affected by various factors that modify the action of the extract upon administration and the subject's biological response to the extract, e.g., severity of lameness, type of tissue damage, the subject's age, sex, and diet, the severity of inflammation, time of administration, and other clinical factors.

Therapeutically effective amounts for administration to a human subject can be determined in animal tests and any art-accepted methods for scaling an amount determined to be effective for an animal for human administration. For example, an amount can be initially measured to be effective in an animal model (e.g., to achieve a beneficial or desired clinical result). The amount obtained from the animal model can be used in formulating an effective amount for humans by using dose conversion factors known in the art. The effective amount obtained in one animal model can also be converted for another animal by using suitable conversion factors such as, for example, body surface area factors.

It is to be understood that, for any particular subject, specific dosage regimes should be adjusted over time according to the individual need and the professional judgment of the person administering or supervising the administration of the washed platelet extract. For example, dosage of the washed platelet extract for a particular subject with lameness can be increased if the lower dose does not elicit a detectable or sufficient improvement in lameness. Conversely, the dosage can be decreased if the lameness is treated or eliminated.

In some cases, therapeutically effective amounts of washed platelet extract can be determined by, for example, measuring the effects of a therapeutic in a subject by incrementally increasing the dosage until the desired symptomatic relief level is achieved. A continuing or repeated dose regimen can also be used to achieve or maintain the desired result. Any other techniques known in the art can be used as well in determining the effective amount range. Of course, the specific effective amount will vary with such factors as the particular disease state being treated, the physical condition of the subject, the type of animal being treated, the duration of the treatment, route of administration, and the nature of any concurrent therapy.

Following administration of washed platelet extract to an individual subject afflicted by, prone to, or likely to develop a disease or injury described herein, a clinical symptom or feature associated with the disease or injury is observed and assessed for a positive or negative change. For example, for methods of treating lameness in a subject, positive or negative changes in the subject's gait, weight bearing position of affected limbs, active or passive flexion of limbs (also referred to as range of motion), or AAEP score (American Association of Equine Practitioners scale, which grades lameness on a scale of 0-5, see for example aaep.org/horsehealth/lameness-exams-evaluating-lame-horse on the World Wide Web) during or following treatment may be determined by any measure known to those of skill in the art including, without limitation, a lameness locator, pressure mats, pressure or force plate gait analysis, measurement of synovial fluid parameters, radiographs, ultrasound, MRI scan, CT scan, and nuclear scintigraphy. See, for example, Broeckx et al. (“Regenerative therapies for equine degenerative joint disease: A preliminary study,” PLOS ONE, 2014, 9(1):e85917).

As used in this specification and the appended claims, the singular forms “a,” “an,” and “the” include plural references unless the context clearly dictates otherwise. Any reference to “or” herein is intended to encompass “and/or” unless otherwise stated.

The terms “comprising”, “comprises” and “comprised of” as used herein are synonymous with “including,” “includes” or “containing,” “contains,” and are inclusive or open-ended and do not exclude additional, non-recited members, elements, or method steps. The phraseology and terminology used herein is for the purpose of description and should not be regarded as limiting. The use of “including,” “comprising,” “having,” “containing,” “involving,” and variations thereof, is meant to encompass the items listed thereafter and additional items. In other words, the terms are intended to be non-exclusive or open-ended. For example, a composition, a mixture, a process, a method, an article, or an apparatus that comprises a list of elements is not necessarily limited to only those elements but may include other elements not expressly listed or inherent to such composition, mixture, process, method, article, or apparatus. Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

As used herein, the term “about,” when referring to a value or to an amount of mass, weight, time, volume, concentration, percentage or a physical dimension such as length, width, or diameter, is meant to encompass variations of in some embodiments of +/−20%, in some embodiments of +/−10%, in some embodiments of +/−5%, in some embodiments of +/−1%, in some embodiments of +/−0.5%, and in some embodiments of +/−0.1% from the specified value or amount, as such variations are appropriate to perform the disclosed methods.

It is understood that any numerical value, range, or either range endpoint (including, e.g., “approximately none”, “about none”, “about all”, etc.) preceded by the word “about,” “substantially” or “approximately” in this disclosure also describes or discloses the same numerical value, range, or either range endpoint not preceded by the word “about,” “substantially” or “approximately.”

The present invention has been described in terms of one or more preferred embodiments, and it should be appreciated that many equivalents, alternatives, variations, and modifications, aside from those expressly stated, are possible and within the scope of the invention.

### EXAMPLES

The use of auto/allogeneic PRP and PL has many limitations. First, virtually all of the beneficial morphogens and anti-inflammatory factors in PRP are derived from the platelets contained therein. However, platelets constitute a small percentage (<0.5%) of the volume of PRP [6]. Further, plasma contains complement, immunoglobulins, albumin and/or fibrinogen, all of which render allogeneic PRP prone to immune/allergic response.

To address issues with PRP and PL known in the art, we demonstrate in Examples 1-5 that purified platelets subsequently washed of plasma from which growth factors are extracted allows for the production of a biological product more concentrated in platelet extract (relative to standard PRP or PL) and also devoid of any plasma proteins which may cause an immune reaction. This Washed Equine PLatelet EXtract (WEPLEX) can be further lyophilized without affecting its biological properties. Our data supports the idea that xenogeneic equine platelet-derived extract may serve as an anti-inflammatory and regenerative pharmaceutical.

Platelets and blood plasma derivatives such as PRP and PL have been extensively tested in regenerative medicine and stem cell-based tissue engineering [5, 36]. There are numerous studies, both in vitro and animal models, demonstrating the efficacy of these blood plasma derivatives [21, 37-41]. Unfortunately, the use of these products in clinical settings, mostly with autologous PRP or PL, has shown limited results in dermatology, dentistry, ophthalmology, orthopedics and others [11, 42, 43]. The major constraint, and so the limited efficacy of all the blood plasma derivatives, is the limited availability of adequate growth factors and cytokines released at the injection site [44]. Furthermore, composition variation correlates with age, sex and patient conditions [45]. In addition, the lack of a standardized protocol reduced the possibility to compare and reproduce all the clinical trials. Some effort has been tested in generating PRP and PL from a pool of multiple donors [46].

Here we addressed these limitations of auto/allogeneic PRP and PL in an entirely novel manner. A novel biological product was created with our formulation of WEPLEX: Washed Equine PLatelet EXtract. The platelets were concentrated by centrifugation and washed multiple times with saline solution to remove plasma proteins such as albumin, immunoglobulins, fibrinogen and complement, which decreases the risk of serum sickness following administration. At the end, the pellet of platelets is solubilized and lysed by the addition of a buffer containing detergent. The detergent emulsifies the platelet membranes and also allows the removal of endotoxins and encapsulated viruses. Moreover, we showed the possibility to lyophilized WEPLEX and store the powder for later uses. This novel formulation is permissive for off-the-shelf use of batch-produced platelet extract for use in human and veterinary medicine. As demonstrated in Table 1, WEPLEX produced using 2% Triton-X114 was 266-fold more concentrated than PRP. Lyophilization also allowed for attaining high concentration of platelet extract post-reconstitution by using a smaller volume of diluent (potentially 2000-fold more concentrated than PL by decreasing 10-fold the initial volume). The new formulation would allow injection of active substances in quantities not otherwise achievable in restricted spaces [e.g., ligaments, tendons and joints].

Equine platelets have been chosen over human and other common mammal platelet products derived from bovine, ovine and porcine sources because human platelets are not as easily acquirable in large quantities and multiple tests are required to assure safety from blood borne diseases. Equine platelets are preferentially employed because no transmissible equin-to-human zoonotic disease has been described to date.

After testing multiple detergents, and considering lysis efficiency as the amount of PDGF over total proteins released compared to PL, 2% v/v Triton X-114 was chosen. Moreover, Triton X-114 has a relatively low clouding point at 22° C. and can be efficiently removed from the final preparation by phase separation. Indeed, centrifugation 15,000×g at 27° C. allowed the separation of the detergent phase (lower phase) with the aqueous/protein phase (upper phase). The detergent phase allowed the removal of endotoxin from the final proteins extract [47, 48].

Denaturation of proteins or loss of biological activity can occur by heating, freezing, agitation in aqueous solutions. Furthermore, storage of liquid solutions of proteins can be quite troublesome. Lyophilization can protect against some of these effects and can extend the shelf life of protein preparations. Under vacuum (very low pressure), the vapor-liquid equilibrium of the solvent is shifted towards the gas phase, while our protein extract, remains primarily in the solid phase. Using vacuum, we easily removed solvent with very little stress on WEPLEX, leaving a dry, solid sample. The dry powder was easily reconstituted in deionized water.

In vitro suppression assay showed that lyophilized/reconstituted WEPLEX was substantially equivalent to a freshly-generated WEPLEX preparation. indeed, with equine or human PHA-activated T cells, both lyophilized/reconstituted or freshly generated WEPLEX were able to suppress T cell proliferation in vitro.

We further tested the immunosuppressive properties of WEPLEX on isolated peritoneal macrophages. Depending on the microenvironment, macrophages can polarize to M1 (inflammatory) or M2 (anti-inflammatory) phenotypes. We established the conversion to an M2-like phenotype by detecting the IL-10 secreted in the media and by establishing the amount of IL-10⁺ cells by flow cytometry. We used peritoneal macrophages derived from Vert-X IL-10 reporter mice. Vert-X mouse cells that produce IL-10 become green and can be easily detected by flow-cytometry. WEPLEX was able to polarize peritoneal macrophages to a M2-immunosuppressive phenotype.

Finally, we determined the regenerative and protective properties of WEPLEX in an acute tissue injury model: DSS-induced colitis in mice. WEPLEX was able to protect the mice from any major weight loss induced by DSS and to increase survival by at least 5 days compared to the mice treated with PBS. These results indicate that WEPLEX has a protective effect in an acute injury model and it can be used in a xenogeneic setting.

The WEPLEX preparations described herein are likely to have a broad and significant impact on acute tissue injury in the human medical field. It could potentially be used not only in acute tissue injury syndromes and regenerative medicine, but also in other medical fields where platelet-derived products have been used, such as dermatology, cardiology and sports medicine. WEPLEX could also have a positive impact in the veterinary medical field where platelet-derived products are used as well for years [49-51].

### Example 1

The embodiment described here demonstrates the production and characterization of a washed equine platelet extract.

### Methods

Whole blood was collected by phlebotomy from the jugular vein from 14 horses (range, 1-33 years; mean 21 years; 12 mares, 8 geldings, 8 Quarter, 2 Peruvian paso, 1 Paint, 1 Belgian Warmblood, 1 Haflinger and 1 Appaloosa) housed at the School of Veterinary Medicine, University of Wisconsin-Madison. CPDA-1 anticoagulant was included in the collection bag to prevent coagulation. Each unit consists of a PL 146 Plastic primary container with 63 mL of CPDA-1 solution containing 2 g dextrose (monohydrate) USP, 1.66 g sodium citrate (dihydrate) USP, 188 mg citric acid (anhydrous) USP, 140 mg monobasic sodium phosphate (monohydrate) USP and 17.3 mg adenine USP, to prevent coagulation Two liters of blood per donation per horse was collected.

After obtaining the whole blood, the sample was transferred into 250 mL conical tubes, under sterile conditions. The undiluted blood was centrifuged at 800×g for 5 min at room temperature (22 to 27° C.), with no brake applied. After the spin, three distinct layers were observed: a bottom layer of red blood cells (accounting for 50-80% of the total volume); a very thin band of white blood cells in the middle layer (also called “buffy coat”) and a top layer of straw-colored platelet-rich plasma (PRP). After centrifugation, the PRP above the red blood cell layer was transferred into new tubes. A second centrifugation at 800×g for 20 min at room temperature allows the isolation of the platelets from the plasma (FIG. 1A). The supernatant is discarded and the pelleted platelets were washed twice with Dulbecco's phosphate-buffered saline (DPBS) without calcium or magnesium. The pelleted platelets can be stored at −80° C. prior to lysis.

Storage of the plasma at 4° C. for between 1 to 7 days resulted in progressive loss of aggregation properties by the platelets. As a result, pelleted platelets are more easily resuspended and washed. Overall lysis yield in the freshly pelleted platelets was lower compared to the platelets left in serum for a few days, most likely due to the aggregates formed during the second centrifugation in fresh plasma. Indeed, it is known that platelets lose aggregation properties over time.

To prepare platelet lysate, different detergent-containing solutions were tested. Lysis buffer composition was assessed by testing different % v/v of detergent in DPBS (Triton X-100, Triton X-114, NP-40, TWEEN-20 and CHAPS at concentration up to 2% v/v). To maximize the concentration density of the platelet extract, the smallest volume of lysis solution that allowed full solubilization of the platelets was added to the washed platelet concentrate for 30-60 min at room temperature. After lysis, the insoluble material was removed by centrifugation at 1000×g at 4° C. Removal of detergent was achieved on detergent removal spin columns, DetergentOUT™, from G-Biosciences following manufacturer's specifications.

In some embodiments of the washed equine platelet extract, the platelets were lysed using 2% Triton-X114 in PBS overnight. Centrifugation at 4000×g allowed the removal of insoluble components from the platelet extract (FIG. 1B). Further centrifugation at 15,000×g at 27° C. removed most of the detergent. Less than 0.1% residual detergent remained. Further scrubbing of the detergent could be achieved using hydrophobic interaction chromatography (HIC).

The biochemical attributes of the different lysed platelet extracts were tested for total protein content and platelet-derived growth factor (PDGF) concentration and the presence of plasma proteins such as total equine IgG (USBiological, Life Sciences) by ELISA, fibrinogen by mechanical clot detection/Stago Compact Maxand (Marshfield Labs) and albumin measured at University of Wisconsin Veterinary Care, Diagnostic Pathology Services. The presence of endotoxin was evaluated by LAL Chromogenic Endotoxin Quantitation Kit (Pierce) in every final lysate prior to in vitro cell culture experiments.

Speed-vac from Eppendorf was used to lyophilize the sample. The sample was placed in 1.5 mL Eppendorf tubes with open cap and freeze on dry ice prior using in speed-vac to prevent loss of material from bumping. Under low pressure vacuum (760 to 25 Torr), the vapor-liquid equilibrium of the solvent is shifted towards the gas phase, while the proteins and molecules of WEPLEX remains in the solid phase. After all the solvent was removed by the process, a solid dry sample remained (plus salts that were present in the solvent buffer). The sample was resuspended in the desired amount of buffer or water.

All statistical analyses were performed using Prism 8 Software. Unless otherwise indicated, tests of statistical significance were conducted using a two-tailed Student's t test. Data display normal variance. P values<0.05 were considered to be statistically significant.

### Results

Two liters of equine whole blood were collected from each horse by phlebotomy at the School of Veterinary Medicine, University of UW-Madison in CPDA-1 bags. Undiluted whole blood was transferred to a 250 mL conical tube under sterile conditions and centrifuged at 800×g for 5 min at room temperature. This low speed centrifugation allows the separation of erythrocytes and leukocytes from the platelet rich plasma (PRP). Two distinct layers could be observed: bottom layer, red blood cells and granulocytes (accounting for 50-80% of the total volume), and a top layer, straw-colored PRP. After centrifugation, the PRP above the red blood cell layer was transferred into new tubes. A second centrifugation at 800×g for 20 min at room temperature allows the separation of the platelets (as a pellet) from the plasma. The supernatant is discarded and the pelleted platelets were washed twice with Dulbecco's phosphate-buffered saline (DPBS) without calcium or magnesium to remove any residual plasma. Pelleted platelets are stored at −80° C. for later processing.

The next step involved platelet disruption with a detergent that would allow for maximal yield of protein content (PDGF served as a surrogate) and could serve as a contemporaneous viral inactivation step (for enveloped viruses). In order to identify an optimal lysis buffer, the proteins extraction efficiency from the platelets was tested with an array of detergents amenable to latter good manufacturing process (GMP) processing (Table 1). We tested: Triton X-114 (0.5-2%), CHAPS (1-0.1%), NP-40 (0.1-2%), TWEEN 20 (0.1-2%) in DPBS. Table 1 shows the optimal percentage v/v for every detergent tested. Pelleted platelets derived from 100-200 mL of PRP were lysed for 1-2 hours at 4° C. and insoluble components were removed by centrifugation 4000×g at 4° C. Removal detergents from extracts was carried out with DetergentOUT™ spin column following manufacturer's instructions.

Following detergent removal, total proteins content were measured by BCA assay. ELISA for PDGF content served as a surrogate of platelet extraction efficiency and ELISA for total IgG content served as a surrogate of residual plasma protein contamination (Table 1). Yield efficiency was calculated as amount of PDGF per total protein respect to the one obtained from PRP (Table 1). Amongst tested detergents, Triton X-114 2% was most efficient in yielding a protein rich platelet extract 266-fold higher than PRP (Table 1). Equine IgG were detected only in the PRP (150±5 mg/mL) and no trace of IgG could be detected in the extracts derived from washed platelets, independently of the lysis detergent used. Similarly, no albumin or fibrinogen could be detected in the equine platelets extract generated with 2% Triton X-114 (Table 1). No endotoxin was detected in the final preparations (detection limit of the kit was 0.06 endotoxin units (EU)/mL).

TABLE 1
PDGF and protein yield efficiency of the washed equine platelet extract using various detergents

| | mg/mL | ng/mL | ng/mL | mg/dL | g/dL | ng PDGF/ | Yield |
|---|---|---|---|---|---|---|---|
| Detergent | protein^{a} | PDGF^{b} | IgGs^{c} | Fibrinogen^{d} | Albumin^{e} | mg protein | efficiency |
| PRP | 11.5 ± 2.8 | 2.3 ± 1.3 | 150 ± 5 | 105 ± 9 | 1 ± 0 | 0.2 ± 0.1 | 1 |
| CHAPS 1% | 1.4 ± 0.2 | 17.1 ± 2.0 | N.D. | N.M. | N.M. | 12.2 ± 2.3 | 61 ± 33 |
| TWEEN 20 1% | 2.0 ± 0.2 | 15.0 ± 2.9 | N.D. | N.M. | N.M. | 7.5 ± 2.6 | 38 ± 22 |
| NP40 1% | 1.6 ± 0.1 | 21.5 ± 3.4 | N.D. | N.M. | N.M. | 13.4 ± 2.3 | 67 ± 35 |
| Triton X-1 14 2% | 1.3 ± 0.3 | 69.2 ± 6.0 | N.D. | N.D. | N.D. | 53.2 ± 13.1 | 266 ± 133 |

^{a}Protein concentration detected with BCA Protein Assay.
^{b}Total equine IgGs and
^{c}PDGF concentration detected by ELISA.
^{d}Detected at Marshfield Labs and
^{e}detected at University of Wisconsin Veterinary Care, Diagnostic Pathology Services.
N.M. not measured,
N.D. not detected
Data represent mean ± SD of 3 different preparations.

### Example 2

The embodiment described herein demonstrates the immunosuppressive properties of the washed equine platelet composition using mouse peritoneal macrophages.

Peritoneal macrophages are non-adherent cells in situ and when they are isolated from the peritoneal cavity and cultured in dishes, they become adherent so that macrophages may be separated from other types of cells from the peritoneal cavity. Peritoneal macrophages are easy to obtain and are therefore used in the art as primary macrophages for in vitro studies. Moreover, depending on the microenvironment, macrophages can polarize to M1 (inflammatory) or M2 (anti-inflammatory) phenotypes.

When macrophages are polarized to an M2-like phenotype they secrete IL-10, one of the key cytokines preventing inflammation-mediated tissue damage. In order to investigate the immunosuppressive properties of the washed equine platelet composition, ELISA assay was used to test the composition's capacity to convert resting macrophages to an M2-like phenotype and produce IL-10 using cells from IL-10 reporter mice. The IL-10 reporter mice used are Vert-X mice, which exhibit baseline intracellular GFP expression in leukocytes via an internal ribosomal entry site-enhanced green fluorescent protein fusion downstream of exon 5 of the IL-10 gene and are therefore useful to the study of IL-10 expression and regulation.

### Methods

Mice (C57BL/6 or Vert-X, either female or male 8-12 weeks old) were anesthetized with isoflurane and peritoneal cells were immediately isolated by washing the peritoneal cavity with sterile DPBS containing 10% FBS. To obtain monolayers of peritoneal macrophages, the cell concentration was adjusted to 5-10 million cells/mL in complete R10 media. The cells were allowed to adhere to the plate by culturing them 16 h at 37° C. Nonadherent cells were then removed by gently washing a few times with warm PBS. Complete R10 was added to the tissue plate with the addition of a range of WEPLEX (0.01-0.1% v/v) for 24 h.

Expression of IL-10 secreted in the culture media was measured by ELISA (Invitrogen). Expression of IL-10 by flow cytometry was carried using peritoneal macrophages from GFP/IL-10 reporter mice (Vert-X mice) and by staining for CD45 and CD11 b antibodies (Invitrogen).

Peritoneal exudate cells from Vert-X mice were plated in six-well plates overnight at 37° C., and the non-adherent cells were removed the day after by replacing the medium and supplementing it with 0.2-0.02-0.02% (v/v) of the washed equine platelet composition. After 24 hours culture medium was tested for IL-10 secretion by anti-mouse IL-10 ELISA and cells were stained for CD45 and CD11b and screened for expression of GFP (IL-10) by flow cytometry. As positive control one well of macrophages was treated with LPS for 24 h.

### Results

Monocytes/Macrophages are innate immune effectors which can be polarized to an M2 immune suppressor state functionally characterized by their production of IL-10 [33]. We tested the ability of WEPLEX to convert resting peritoneal macrophages to an IL-10⁺ M2-immunosuppressive like phenotype. To assess M2 polarization, we utilized peritoneal macrophages derived from Vert-X mice [34] which co-express GFP when IL-10 expression takes place.

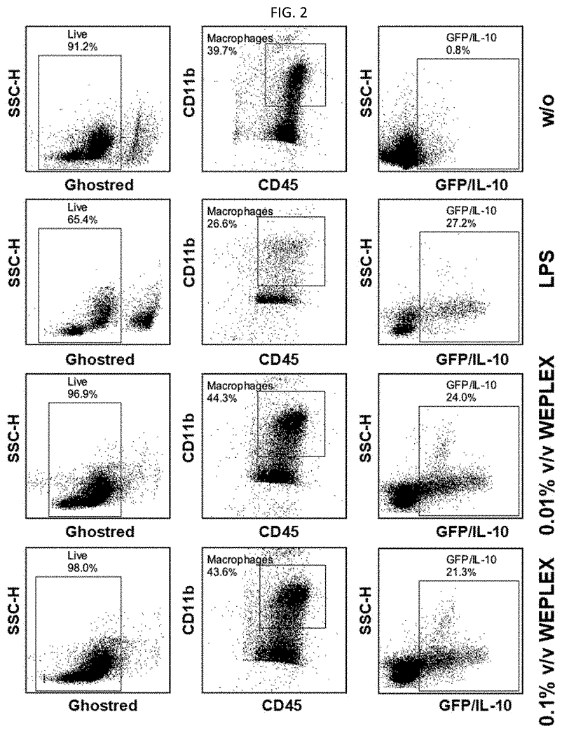

Peritoneal cells from Vert-X mice were plated in six-well plates overnight at 37° C., and the non-adherent cells were removed the day after by replacing the media and supplementing it with 0.1-0.01% v/v of WEPLEX. After overnight culture, the overlying media was tested for IL-10 secretion by ELISA and cells were stained for CD45 and CD11 b and screened for the co-expression of GFP (IL-10) by flow cytometry (FIG. 2). As a positive control for macrophage activation, macrophages was treated with LPS for 24 h. Macrophages treated with LPS secreted in the culture media the highest amount of IL-10 (1.1 ng/mL). Macrophages treated with 0.01% or 0.1% v/v WEPLEX secreted 0.3 ng/mL of IL-10 while. These cells also displayed an increase frequency of GFP⁺/IL10⁺ macrophages (21.2% CD11b⁺CD45⁺GFP⁺ for 0.01% v/v of WEPLEX, FIG. 2). IL-10 could not be detected in the macrophages cultured in complete media without WEPLEX.

### Example 3

The embodiment described herein demonstrates the safety of washed equine platelet composition. Also described in this embodiment is the effect of the washed equine platelet composition on dextran sulfate sodium (DSS)-induced colitis (colon inflammation) in mice.

### Methods

Safety—To evaluate safety of the final formulation of WEPLEX, one group of mice (n=3) received 1 mL of WEPLEX intraperitoneally (IP) every other day for 2 rounds of injections. Signs for sensitization were monitored immediately post-injection and over time. None of the mice sensitized with the 1 mL dose showed signs of labored breathing, lethargy, tremors, or other signs of shock when challenged with WEPLEX.

Soft-tissue injury model—DSS-induced acute colitis model was used to explore the therapeutic and possibly regenerative effect of the washed equine platelet extract in mice. DSS with MW 40-44 kDa (Sigma) was dissolved in ultra-pure water at concentrations of 4%. C57BL/6 (either female or male 8-12 weeks old) mice were exposed to the DSS solutions ad libitum for 7 days followed by regular water thereafter. At day 2 and day 4 mice received either 1 mL of the washed equine platelet extract or PBS intraperitoneally (IP). Mice treated with DSS lose a significant amount of body weight as sign of the disease. A weight loss larger than 25% of the initial body weight is considered the end-point for the animal by IACUC protocol standards.

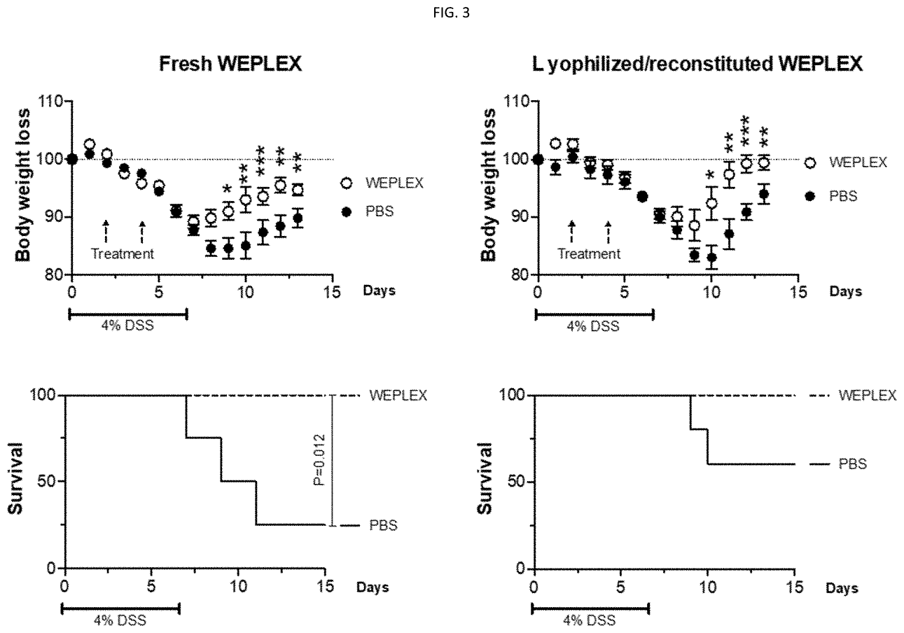

IP administration of washed equine platelet extract protected the mice from any major weight loss induced by DSS (see FIG. 3). These findings demonstrate that the washed equine platelet extract has a protective effect in an acute injury model. In addition, it showed that the washed equine platelet extract can be used in a xenogeneic setting (equine derived platelet product injected in a mouse model).

### Results

Provision of DSS in drinking water leads to toxic colitis manifest clinically by weight loss [35]. We used this acute tissue injury model to test the anti-inflammatory pharmaceutical properties of WEPLEX administered parenterally. Fresh WEPLEX was prepared and lyophilized WEPLEX was reconstituted in the same volume from which it was generated. To induce colitis, DSS (molecular weight 5,000; 1VIP Biomedicals Inc., Eschwege, Germany) was added into drinking water C57BL/6 mice (either male or female, 8-12 weeks old) at a concentration of 4% (w/v) for 7 days. After 7 days, DSS water was exchanged for fresh water. Mice were randomly allocated into four groups (n=5 each) as follows: WEPLEX group was given 1 mL of fresh extract or lyophilized/reconstituted intra-peritoneal (IP) on day 2 and 4, control groups were given 1 mL of PBS on day 2 and 4. The IP administration of WEPLEX was able to significantly mitigate weight loss during the peak of the colitic syndrome around day 10 (FIG. 3, upper panels), and also improved overall survival in mice (FIG. 3, lower panels).

### Example 4

In vitro suppression assays are used to determine the suppressive capacity of cells or compounds by measuring their abilities to suppress the proliferation of responding T cells. The responder cells are labeled with a proliferation tracking dye or an antibody. The responder cells are incubated with the cells or compounds and stimulated for proliferation in the presence of immunomodulatory compounds. The proliferation of the responder cells in each treatment condition is tracked by flow cytometry after 3-4 days of activation.

### Methods

Equine and human peripheral blood mononuclear cells (PBMCs) were isolated by the modified protocol described by Panda et al. [30]. Briefly, anticoagulant-treated blood, diluted two-fold on DPBS, was carefully layered on top of one-volume of Ficoll-Paque PLUS density gradient media (GE Healthcare) in a tube. The tube was then centrifuged 400×g for 30 min at 25° C. This first centrifugation step generated three layers: the upper layer was plasma, the intermediate layer containing mononuclear cells and the bottom layer, which consisted mostly of erythrocytes and polymorphonuclear granulocytes. The plasma/Ficoll medium interface contains both PBMCs and platelets. To isolate PBMCs, this interface was carefully removed, washed with a salt-buffered solution, and then centrifuged. The supernatant, containing platelets, Ficoll and plasma, was removed, leaving a pellet of purified PBMCs.

PBMCs were cultured in 12 or 24 tissue plates (Corning®) at a density of 1 million cells/mL in complete R10: RPMI-1640 with L-Glu (Corning), 10% fetal bovine serum -FBS (Sigma), 2 mM L-Glutamine (Lonza), 10 mM HEPES (Lonza), 0.1 mM Beta-mercaptoethanol (Gipco) and the addition of 100 U/mL penicillin/streptomycin.

T cells activation was achieved by stimulating T cells with phytohemagglutinin-L (PHA-L), Invitrogen™ [31]. Cells were cultured 3 days with PHA-L solution (500×) at 2 μL per mL of culture medium.

In our assay, we tested the suppression of phytohemagglutinin (PHA)-activated T cells with WEPLEX. We isolated peripheral blood mononuclear cells (PBMCs) from whole equine blood or whole human blood.

The proliferation of equine PBMCs was tested by labeling the cells with carboxyfluorescein succinimidyl ester (CFSE). CFSE is a cell-permeant, non-fluorescent pro-dye. Intracellular esterases in live cells cleave the acetate groups of CFSE producing a molecule with green fluorescence that is also now membrane impermeant. The succinimidyl ester group reacts indiscriminately with intracellular free amines to generate covalent dye-protein conjugates. The result is live cells with an intracellular fluorescent label. The content of CFSE decreases in proliferating cells per generation.

Equine PBMCs were labeled with 5 μM carboxyfluorescein succinimidyl ester (CFSE; Molecular Probes, Eugene, Oreg., USA) in 1mL of PBS for 15 min at 37° C. The labeling was halted by adding an excess of fetal calf serum (FCS), and the samples were washed twice with RPMI-1640 (Corning) supplemented with 10% FBS (Sigma). The CFSE-labeled cells were cultured with a range of WEPLEX (0.01-10% v/v), fresh or freshly reconstituted lyophilized. The T cells CFSE fluorescence intensity was analyzed by flow cytometry after 3-4 days of co-culture and proliferation. The analysis of CD8 T cells was performed with the use of anti-equine CD8 (Invitrogen) flow cytometry antibodies.

For human T cells proliferation, PBMCs were labeled post-treatment with Ki-67 (also known as MKI67) a cellular marker for proliferation. Ki-67 is strictly associated with cell proliferation. During interphase, the Ki-67 antigen can be exclusively detected within the cell nucleus, whereas in mitosis most of the protein is relocated to the surface of the chromosomes.

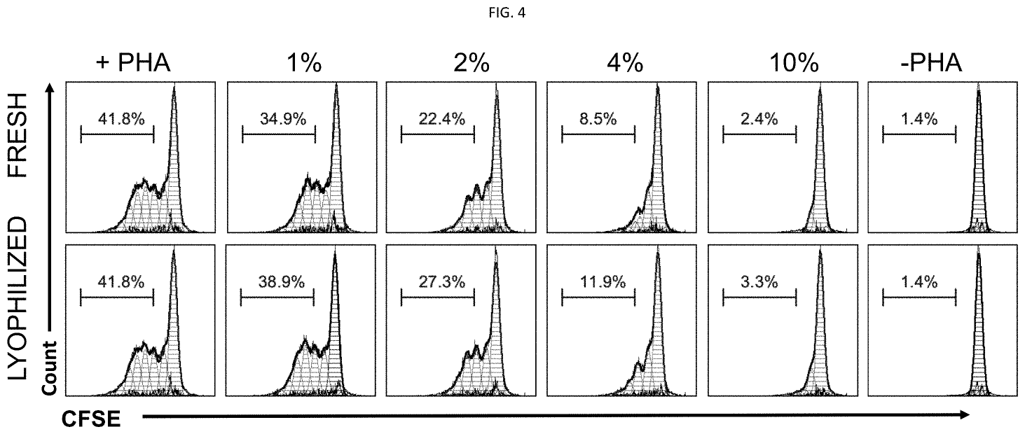

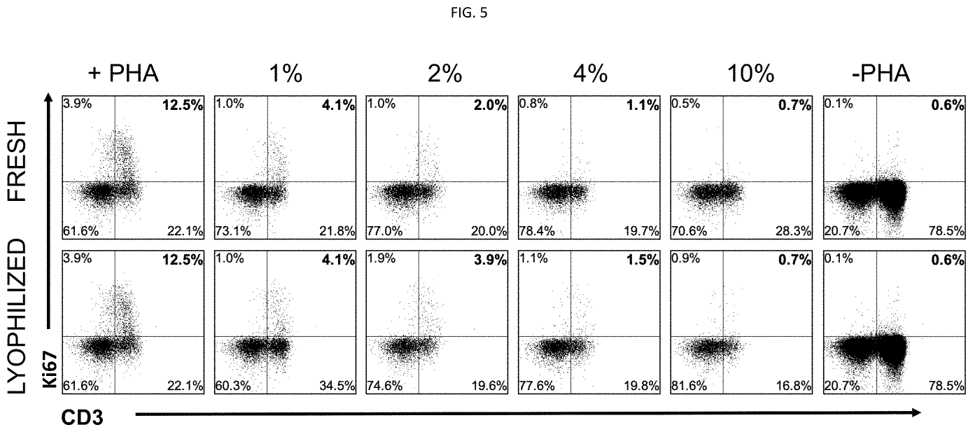

PBMCs, from horse and human, were cultured in the presence of WEPLEX (1-10% v/v) and presence or absence of PHA. After 3 or 4 days the cells were collected, stained with a viable dye followed by surface staining with T cells markers. Proliferation was detected by flow cytometry (FIGS. 4 and 5). Concentrations of WEPLEX up to 10% were able to suppress T cells proliferation, both for equine PBMCs (allogeneic properties), and human PBMCs (xenogeneic properties).

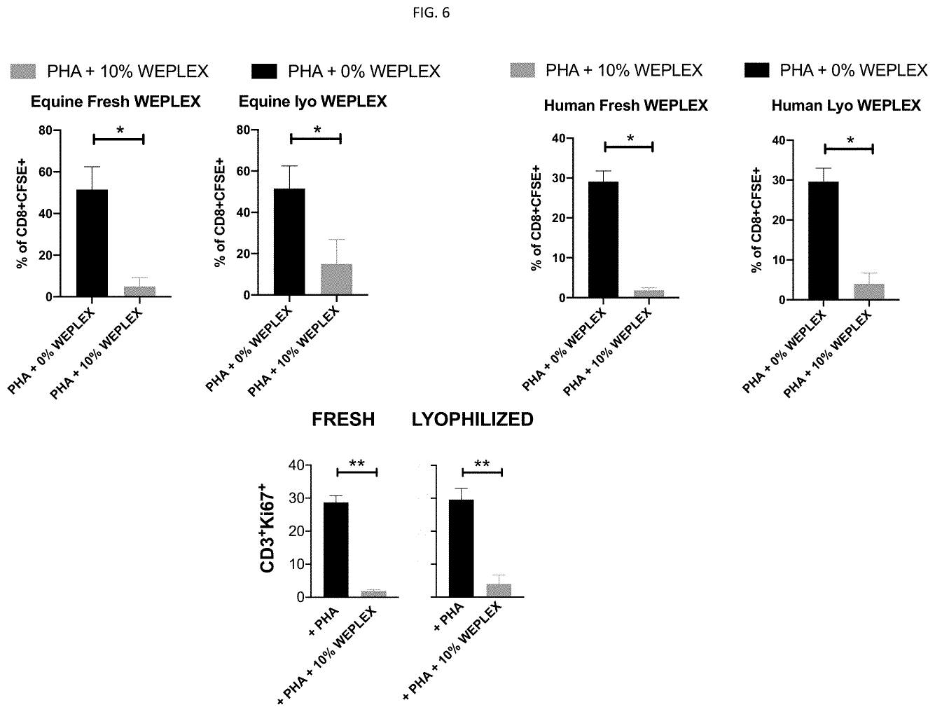

Furthermore, two different formulations of WEPLEX were tested: fresh and lyophilized/reconstitute. To maintain the same potency (in terms of regenerative components of the extract) the lyophilized sample was reconstituted in the same volume from what it was generated (i.e. 1 mL of WEPLEX was lyophilized; 1 mL of molecular biology grade water was used for reconstitution). As shown in FIG. 6, the lyophilized/reconstitute sample has similar bioactivity compared to the freshly prepared. Lyophilization can increase the shelf life of the extract without affecting its potency.

### Results

PRP and related platelet lysate derivatives have been shown to be potent suppressors of T-cell functionality, including TCR-mediated mitogenic response [32]. To determine the T-cell immunosuppressive potential of WEPLEX, we analyzed its ability to suppress the replicative response of equine or human phytohemagglutinin (PHA) activated peripheral blood mononuclear cells (PBMCs). Ficoll-enriched PBMCs from horse and human were cultured in presence of WEPLEX (1-10% v/v) and PHA. After 72-96 hours, the cells were collected and analyzed by flow cytometry (FIGS. 4, 5, and 6). The proliferation of equine PBMCs was tested by labeling the cells with carboxyfluorescein succinimidyl ester (CFSE) prior to stimulation and thereafter assessing dye dilution as a surrogate of replication (FIGS. 4 and 6). Concentration as low as 1% v/v of WEPLEX was able to significantly decrease the proliferation of equine CD8⁺ T cells (41.8% CD8⁺CFSE⁺ proliferating T cells respect 34.9% CD8⁺CFSE⁺ with 1% v/v WEPLEX, FIG. 4). Full suppression of proliferation was achieved with 10% v/v of WEPLEX added to the culture media (2.4% CD8⁺CFSE⁺, FIG. 4). Similar results were observed with the lyophilized/reconstituted WEPLEX formulation (FIGS. 4 and 6).

For human T-cell proliferation, PBMCs were labeled post-PHA activation with Ki-67 (FIGS. 5 and 6). Concentration of 1% v/v of WEPLEX, both with the fresh or lyophilized/reconstituted WEPLEX formulation, decreased the proliferation of human CD3⁺ T cells more than 3-fold with respect to the PHA-stimulated control (12.5% CD3⁺Ki67⁺ proliferating T cells respect 4.1% CD3⁺Ki67⁺ with 10% v/v of WEPLEX, FIGS. 5 and 6). Full inhibition of human T cells proliferation was achieved with concentration of 4% v/v of WEPLEX (0.7 CD3⁺Ki67⁺, FIGS. 5 and 6).

### Example 5

We used the speed-vac to concentrate 1 mL samples of WEPLEX from 3 different preparations. Under vacuum (very low pressure), the vapor-liquid equilibrium of the solvent is shifted towards the gas phase, while our sample, WEPLEX, remains primarily in the solid phase. Using vacuum we easily removed solvent with very little stress on WEPLEX, leaving with a dry, solid sample (plus salts that were present in the solvent buffer, PBS). The powder was stored at room temperature and reconstitute in water prior to use. Filter sterilization was used to sterilize the solution since the lyophilization process was not carried out under controlled environment (i.e., a biosafety cabinet).

Lyophilization of WEPLEX would provide for extended shelf life assuming potency quality attributes are preserved. To test the effect of lyophilization on WEPLEX, we dehydrated 1 mL aliquote of WEPLEX by freezing and drying in the Eppendorf's speed-vac. Usually, 1 mL aliquote required 5 h to be fully lyophilized. Throughout the process, the WEPLEX solution remained clear and without any precipitate. The final rendered powder was stored at room temperature and reconstitute in 1 mL of deionized water prior to testing. Microporous filtration with a membrane with 0.2 μm pore size was used to maintain sterility and prevent contamination post-lyophilization.

In order to establish the composition of the two different formulations of WEPLEX, fresh and lyophilized, total protein and PDGF content have been established by BCA and ELISA, respectively. Table 2 shows a comparison of fresh and lyophilized/reconstitute WEPLEX by protein concentration and PDGF detected. WEPLEX was generated as usual and divided in two different tubes: one was kept at 4° C. and the other process in speed-vac to be lyophilized. The day after the lyophilized samples were reconstituted in deionized water and filter sterilized. Table represent 2 independent batches of WEPLEX, mean ±SEM

TABLE 2
Comparison of fresh and lyophilized/reconstituted WEPLEX

| Sample | mg/mL protein^{a} | ng/mL PDGF^{b} | ng PDGF/mg protein |
|---|---|---|---|
| Fresh | 3.5 ± 0.7 | 87 ± 1 | 25 ± 1 |
| Lyophilized/ | 3.1 ± 0.8 | 78 ± 5 | 25 ± 7 |
| reconstituted | | | |

^{a}Protein concentration measured with BCA Protein Assay Kit.
^{b}PDGF concentration detected by ELISA.
Freshly prepared WEPLEX was divided in two different aliquots. One aliquot was lyophilized and the other kept at 4° C. The day after the lyophilized sample was reconstituted in deionized water and filter sterilized. Protein concentrations and PDGF content were measured. Data represent mean ± SD of 3 different preparations.

No significant differences in protein concentrations and PDGF were detectable between the two different formulations based on biochemical composition. Moreover, the powder can be reconstituted in a much smaller volume than the initial volume size lyophilized, thus increasing the potency of the extract by at least an order a magnitude. The bioactivity of the fresh and lyophilized formulations was tested as per Example 4.

### REFERENCES

1. Scully D, et al., Platelet biology in regenerative medicine of skeletal muscle. Acta Physiol (Oxf). 2018; 223(3):e13071. Epub 2018 Apr. 11.
2. Etulain J. Platelets in wound healing and regenerative medicine. Platelets. 2018; 29(6):556-68. Epub 2018 Feb. 15.
3. Holinstat M. Normal platelet function. Cancer Metastasis Rev. 2017; 36(2):195-8. Epub 2017 Jul. 2.
4. Santos S, et al., Blood Plasma Derivatives for Tissue Engineering and Regenerative Medicine Therapies. Tissue Eng Part B Rev. 2018; 24(6):454-62. Epub 2018 May 9.
5. Burnouf T, et al., Blood-derived biomaterials and platelet growth factors in regenerative medicine. Blood Rev. 2013; 27(2):77-89. Epub 2013 Feb. 26.
6. Alves R, et al., A Review of Platelet-Rich Plasma: History, Biology, Mechanism of Action, and Classification. Skin Appendage Disorders. 2018; 4(1):18-24.
7. Conde Montero E, et al., Platelet-rich plasma: applications in dermatology. Actas Dermosifiliogr. 2015; 106(2):104-11. Epub 2014 May 6.
8. Patel A N, et al., Evaluation of autologous platelet rich plasma for cardiac surgery: outcome analysis of 2000 patients. J Cardiothorac Surg. 2016; 11(1):62. Epub 2016 Apr. 14.
9. Mihaylova Z, et al., Use of platelet concentrates in oral and maxillofacial surgery: an overview. Acta Odontol Scand. 2017; 75(1):1-11. Epub 2016 Sep. 28.
10. Dawood A S, et al., Current clinical applications of platelet-rich plasma in various gynecological disorders: An appraisal of theory and practice. Clin Exp Reprod Med. 2018; 45(2):67-74. Epub 2018 Jul. 10.
11. Zhang M, et al., Applications and efficacy of platelet-rich plasma in dermatology: A clinical review. J Cosmet Dermatol. 2018; 17(5):660-5. Epub 2018 Jul. 27.
12. Copland I B, et al., The effect of platelet lysate fibrinogen on the functionality of MSCs in immunotherapy. Biomaterials. 2013; 34(32):7840-50. Epub 2013 Jul. 31.
13. Laner-Plamberger S, et al., Mechanical fibrinogen-depletion supports heparin-free mesenchymal stem cell propagation in human platelet lysate. J Transl Med. 2015; 13:354.
14. Jo C H, et al., ALLOGENEIC PLATELET-RICH PLASMA FOR ROTATOR CUFF REPAIR. Acta Ortop Bras. 2017; 25(1):38-43.
15. Le A D K, et al., Current Clinical Recommendations for Use of Platelet-Rich Plasma. Curr Rev Musculoskelet Med. 2018; 11(4):624-34. Epub 2018 Oct. 26.
16. Alio J L, et al.,The role of “eye platelet rich plasma” (E-PRP) for wound healing in ophthalmology. Curr Pharm Biotechnol. 2012; 13(7):1257-65. Epub 2011 Jul. 12.
17. Suthar M, et al., Treatment of chronic non-healing ulcers using autologous platelet rich plasma: a case series. J Biomed Sci. 2017; 24(1):16. Epub 2017 Mar. 1.
18. Calori G M, et al., Application of rhBMP-7 and platelet-rich plasma in the treatment of long bone non-unions: a prospective randomised clinical study on 120 patients. Injury. 2008; 39(12):1391-402. Epub 2008 Nov. 26.
19. Tan X-x, et al., Autologous platelet lysate local injections for the treatment of refractory lateral epicondylitis. J Orthop Surg Res. 2016; 11:17.
20. Giannaccare G, et al., Blood derived eye drops for the treatment of cornea and ocular surface diseases. Transfus Apher Sci. 2017; 56(4):595-604. Epub 2017 Aug. 29.
21. Tambella A M, et al., Platelet-rich Plasma and Other Hemocomponents in Veterinary Regenerative Medicine. Wounds. 2018; 30(11):329-36. Epub 2018 Nov. 13. PubMed PMID: 30418162.
22. Brossi P M, et al., Platelet-rich plasma in orthopedic therapy: a comparative systematic review of clinical and experimental data in equine and human musculoskeletal lesions. BMC Vet Res. 2015; 11:98. Epub 2015 Apr. 22.
23. Ortved K F. Regenerative Medicine and Rehabilitation for Tendinous and Ligamentous Injuries in Sport Horses. Vet Clin North Am Equine Pract. 2018; 34(2):359-73. Epub 2018 May 29.
24. Tambella A M, et al., Platelet-rich plasma to treat experimentally-induced skin wounds in animals: A systematic review and meta-analysis. PLoS One. 2018; 13(1):e0191093. Epub 2018 Jan. 13.
25. Cengiz I F, et al., PRP Therapy. Adv Exp Med Biol. 2018; 1059:241-53. Epub 2018 May 8.
26. Filardo G, et al., Platelet-rich plasma: why intra-articular? A systematic review of preclinical studies and clinical evidence on PRP for joint degeneration. Knee Surg Sports Traumatol Arthrosc. 2015; 23(9):2459-74. Epub 2013 Nov. 28.
27. Burnouf T, et al., Viral safety of human platelet lysate for cell therapy and regenerative medicine: Moving forward, yes, but without forgetting the past. Transfus Apher Sci. 2019:102674. Epub 2019 Nov. 19.
28. Shih D T, et al., Preparation, quality criteria, and properties of human blood platelet lysate supplements for ex vivo stem cell expansion. N Biotechnol. 2015; 32(1):199-211. Epub 2014 Jun. 15.
29. Crespo-Diaz R, et al., Platelet lysate consisting of a natural repair proteome supports human mesenchymal stem cell proliferation and chromosomal stability. Cell Transplant. 2011; 20(6):797-811. Epub 2010 Nov. 26.
30. Lan K, et al., Isolation of human peripheral blood mononuclear cells (PBMCs). Curr Protoc Microbiol. 2007; Appendix 4:Appendix 4C. Epub 2008 Nov. 5.
31. Movafagh A, et al., The Significance Application of Indigenous Phytohemagglutinin (PHA) Mitogen on Metaphase and Cell Culture Procedure. Iran J Pharm Res. 2011; 10(4):895-903. Epub 2011 Oct. 1. PubMed PMID: 24250428; PubMed Central PMCID: PMCPMC3813071.
32. Zamora C, et al., Binding of Platelets to Lymphocytes: A Potential Anti-Inflammatory Therapy in Rheumatoid Arthritis. J Immunol. 2017; 198(8):3099-108. Epub 2017 Mar. 3.
33. Atri C, et al., Role of Human Macrophage Polarization in Inflammation during Infectious Diseases. International journal of molecular sciences. 2018; 19(6):1801.
34. Madan R, et al., Nonredundant roles for B cell-derived IL-10 in immune counter-regulation. J Immunol. 2009; 183(4):2312-20. Epub 2009 Jul. 22.
35. Chinnadurai R, et al., Challenges in animal modelling of mesenchymal stromal cell therapy for inflammatory bowel disease. World J Gastroenterol. 2015; 21(16):4779-87.
36. Bielecki T, et al., Platelet-rich plasma (PRP) and Platelet-Rich Fibrin (PRF): surgical adjuvants, preparations for in situ regenerative medicine and tools for tissue engineering. Curr Pharm Biotechnol. 2012; 13(7):1121-30. Epub 2011 Jul. 12.
37. Borrione P, et al., Experimental model for the study of the effects of platelet-rich plasma on the early phases of muscle healing. Blood Transfus. 2014; 12 Suppl 1:s221-8. Epub 2013 Jul. 23.
38. Marques L F, et al., Platelet-rich plasma (PRP): methodological aspects and clinical applications. Platelets. 2015; 26(2):101-13. Epub 2014 Feb. 12.
39. Sermer C, et al., The Addition of Platelet-Rich Plasma to Scaffolds Used for Cartilage Repair: A Review of Human and Animal Studies. Arthroscopy. 2015; 31(8):1607-25. Epub 2015 Apr. 1.
40. Gianakos A, et al., Platelet-Rich Plasma in the Animal Long-Bone Model: An Analysis of Basic Science Evidence. Orthopedics. 2015; 38(12):e1079-90. Epub 2015 Dec. 15.
41. Tseng CL, et al., Animal models to assess the therapeutic efficacy of human serum and serum-converted platelet lysates for dry eye syndrome: Seeing is believing. Transfus Apher Sci. 2015; 53(1):95-8. Epub 2015 Jun. 23.
42. Laudy AB, et al., Efficacy of platelet-rich plasma injections in osteoarthritis of the knee: a systematic review and meta-analysis. Br J Sports Med. 2015; 49(10):657-72. Epub 2014 Nov. 25.
43. Soni NG, et al., Blood-derived topical therapy for ocular surface diseases. Br J Ophthalmol. 2016; 100(1):22-7. Epub 2015 Jul. 17.
44. Ramaswamy Reddy SH, et al., Stem-cell therapy and platelet-rich plasma in regenerative medicines: A review on pros and cons of the technologies. J Oral Maxillofac Pathol. 2018; 22(3):367-74.
45. Masoudi E, et al., Platelet-Rich Blood Derivatives for Stem Cell-Based Tissue Engineering and Regeneration. Curr Stem Cell Rep. 2016; 2(1):33-42. Epub 2016 Apr. 6.
46. Klatte-Schulz F, et al., Comparative Analysis of Different Platelet Lysates and Platelet Rich Preparations to Stimulate Tendon Cell Biology: An In Vitro Study. Int J Mol Sci. 2018; 19(1). Epub 2018 Jan. 11.
47. Aida Y, et al., Removal of endotoxin from protein solutions by phase separation using Triton X-114. J Immunol Methods. 1990; 132(2):191-5. Epub 1990 Sep. 14.
48. Ma R, et al., Removing endotoxin from plasmid samples by Triton X-114 isothermal extraction. Anal Biochem. 2012; 424(2):124-6. Epub 2012 Mar. 1.
49. Fortier L A, et al., Regenerative medicine for tendinous and ligamentous injuries of sport horses. Vet Clin North Am Equine Pract. 2008; 24(1):191-201.
50. Cheng G, et al., Incorporating platelet-rich plasma into coaxial electrospun nanofibers for bone tissue engineering. Int J Pharm. 2018; 547(1-2):656-66. Epub 2018 Jun. 7.
51. Altaie A, et al., Use of platelet lysate for bone regeneration—are we ready for clinical translation? World J Stem Cells. 2016; 8(2):47-55.
52. Luzo A M C, et al. What is the potential use of platelet-rich-plasma (PRP) in cancer treatment? A minireview. Helivon, 2020:6(3)e03660

1. A method of producing a washed platelet extract comprising the steps of:
i) separating plasma from a whole blood sample;
ii) separating platelets from the plasma;
iii) washing the platelets with buffered saline;
iv) adding a detergent solution comprising between 0.1% and 2.5% detergent to the washed platelets and incubating the washed platelets with the detergent solution for a time sufficient to lyse at least 50% of the platelets, whereby a platelet lysate is formed; and
v) separating the washed platelet extract from insoluble components of the platelet lysate.

2. The method of claim 1, additionally comprising the step of removing residual detergent from the washed platelet extract by spinning or using hydrophobic interaction chromatography.

3. The method of claim 1, wherein in step (i), the plasma is separated from the whole blood sample by spinning the whole blood sample at a speed between 600×g and 1000×g for about 5 minutes at a temperature between 22° C. and 27° C.

4. The method of claim 1, wherein in step (ii), the platelets are separated from the plasma by spinning the plasma at a speed between 600×g and 1000×g for about 25 minutes at a temperature between 22° C. and 27° C.

5. (canceled)

6. The method of claim 1, wherein in step (iii), the platelets are washed at least 2 times.

7. The method of claim 1, wherein the detergent selected from the group consisting of (1,1,3,3-Tetramethylbutyl)phenyl-polyethylene glycol (TRITON™ X-114), nonyl phenoxypolyethoxylethanol (NP-40), 3-[(3-Cholamidopropyl)dimethylammonio]-1-propanesulfonate (CHAPS), or polyoxyethylene (20) sorbitan monolaurate (TWEEN 20).

8. (canceled)

9. The method of claim 1, wherein in step (v), the washed platelet extract is separated from the insoluble components by spinning the platelet lysate at a speed between 3000×g and 5000×g for about 10 minutes at about 4° C.

10. The method of claim 1, additionally comprising the step of lyophilizing the washed platelet extract to form a lyophilized washed platelet extract.

11. A washed platelet extract generated by the method of claim 1, wherein the washed platelet extract is free of plasma serum protein.

12. The washed platelet extract of claim 11 comprising at least 20 ng/mL of platelet growth factors when prepared from 50 mL of plasma.

13. The washed platelet extract of claim 12, wherein the platelet growth factors include platelet derived growth factor (PDGF).

14. The washed platelet extract of claim 11 comprising less than 2 mg/mL total protein when prepared from 50 mL of plasma.

15. A pharmaceutical composition comprising the washed platelet extract of claim 11 and a pharmaceutically acceptable carrier.

16. A method of treating lameness in a horse in need thereof, comprising administering to the horse a therapeutically effective amount of the pharmaceutical composition of claim 15, wherein the whole blood sample is an equine whole blood sample.

17. The method of claim 16, wherein the pharmaceutical composition is administered at a volume between 0.1 mL and 1.0 L.

18. A method of treating tendon or ligament injury in a subject in need thereof, comprising administering to the subject a therapeutically effective amount of the pharmaceutical composition of claim 15.

19. The method of claim 18, wherein the subject is a human, a horse, a dog, a cat, a camelid, or a cow.

20. The method of claim 1, wherein the whole blood sample is an equine whole blood sample and the washed platelet extract produced is a washed equine platelet extract.

21. The method of claim 1, wherein prior to step (ii) the plasma is stored for at least 1 day at 4° C.

22. (canceled)

23. The washed platelet extract of claim 11, wherein the washed platelet extract is lyophilized.
